(19) United States
(12) Patent Application Publication
(10) Pub. No.: US 2012/0271370 A1
(43) Pub. Date: Oct. 25, 2012

(54) Automated external defibrillator locating system and method

(76) Inventors: Keith W. Hochhalter, Inverness, IL (US); Michael T. Seiler, Kenosha, WI (US)

(21) Appl. No.: 13/066,720

(22) Filed: Apr. 22, 2011

(51) Int. Cl. A61N 1/39; G08B 3/10

(52) U.S. Cl. 607/5; 340/8.1

(57) ABSTRACT

A defibrillator locating system and method enables would-be responders to quickly identify defibrillator location for effecting prompt defibrillation. The system may be said to comprise a number of spaced heart-shaped signal-transmitting devices, a signal-receiving/alarm device, and an automatic external defibrillator pair-positioned in visual proximity to the signal-receiving/alarm device. A responder may send a signal from any of the transmitter devices for activating the nearest receiving/alarm device, the latter of which activates an alarm signal for guiding the responder toward the automatic external defibrillator. The alarm signal is preferably auditory and has sufficient intensity whereby the responder may be able to aurally perceive the auditory signal and follow the same to the defibrillator site along an unobstructed pathway. The removably attached defibrillator enables the responder to quickly carry said defibrillator to a cardiac emergency site.

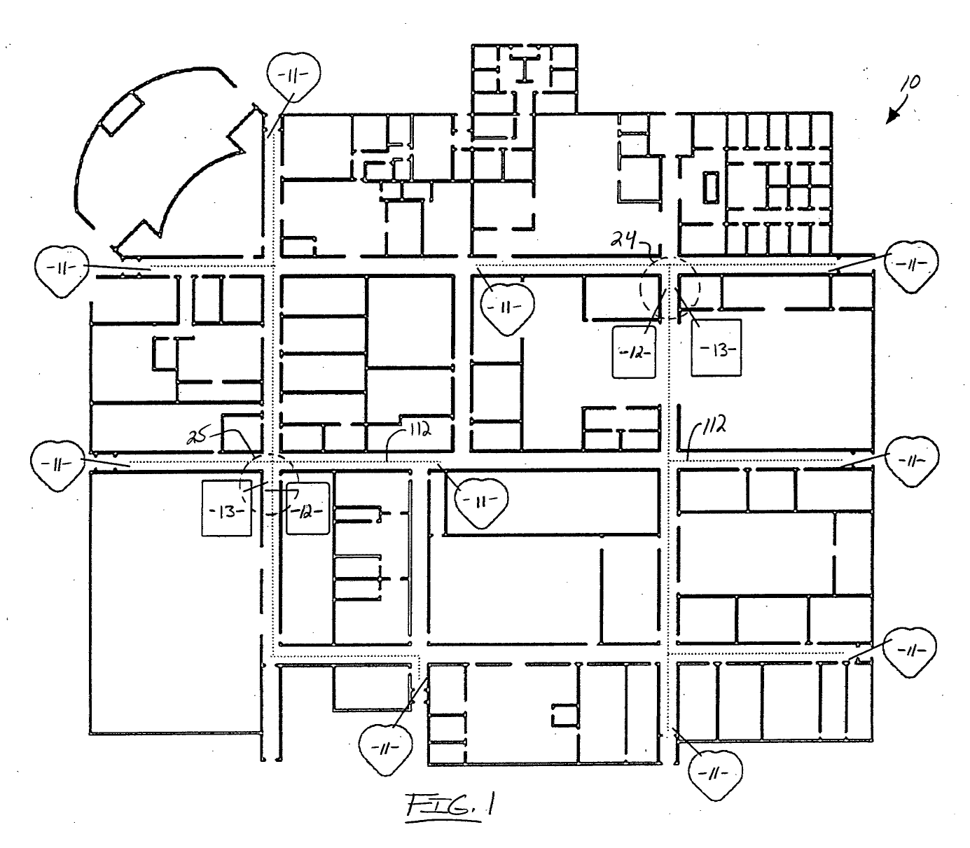

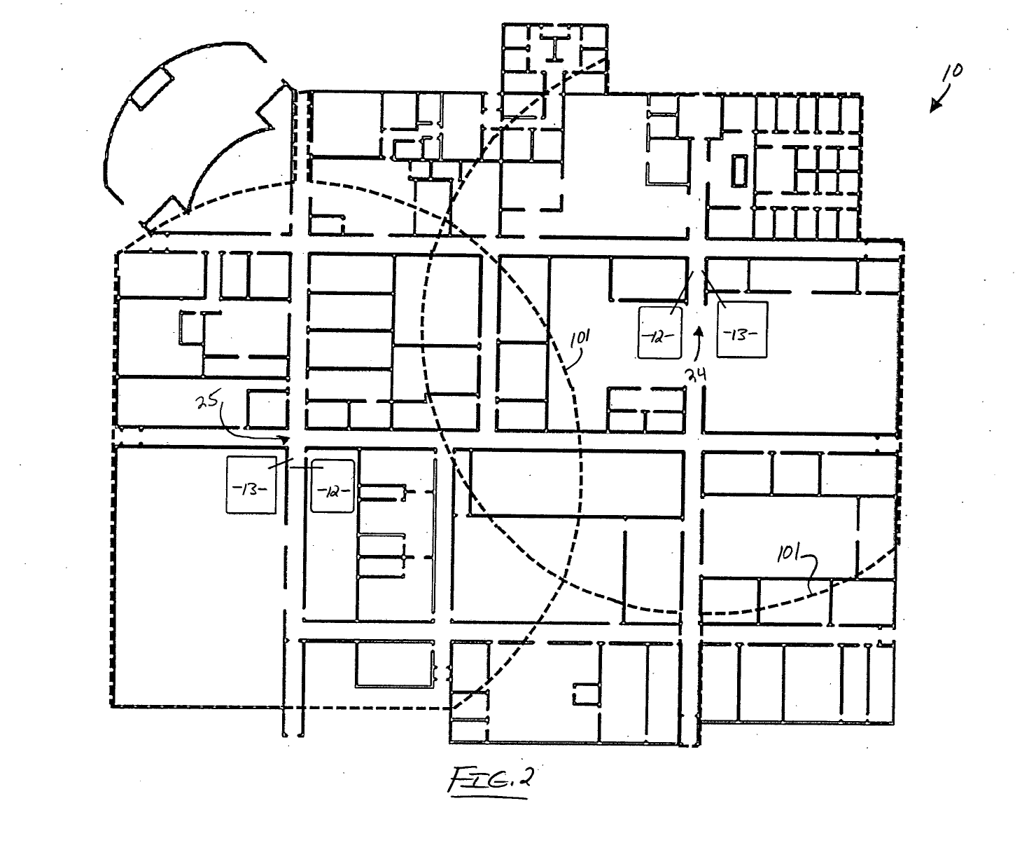

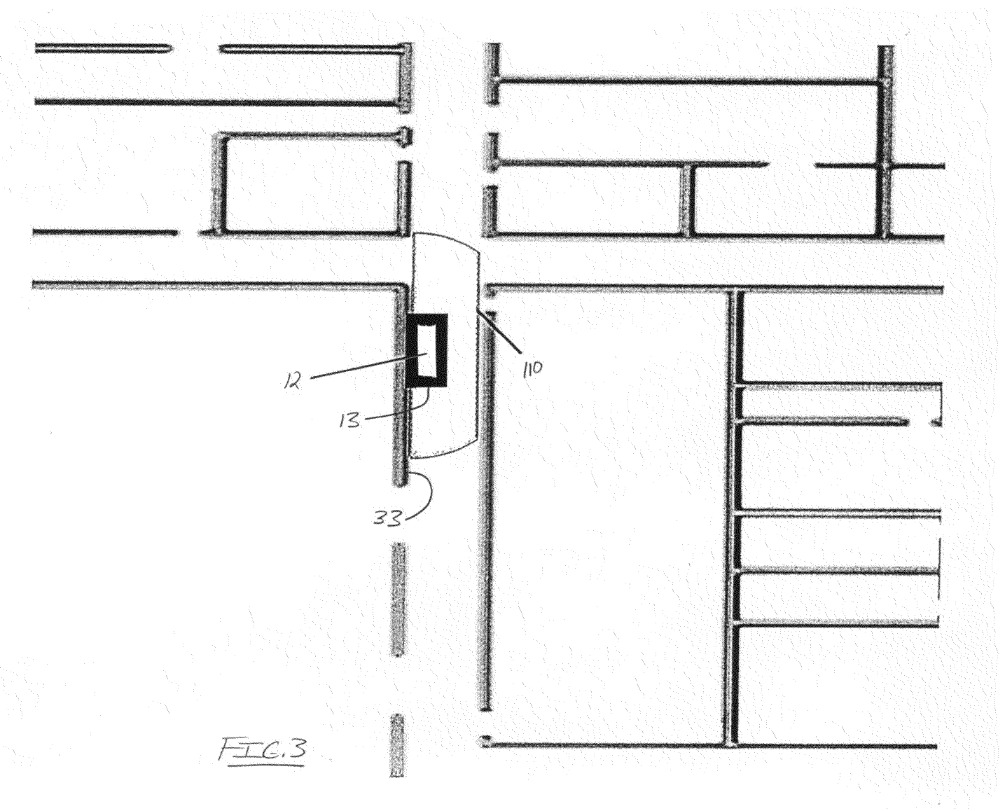

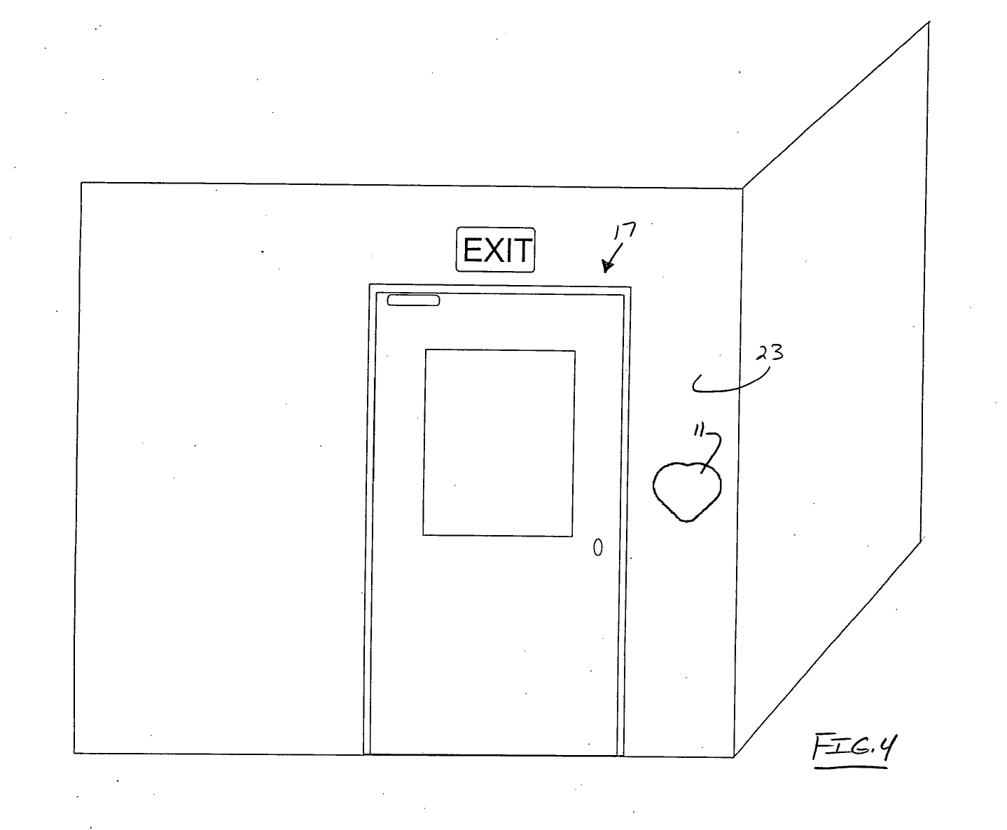

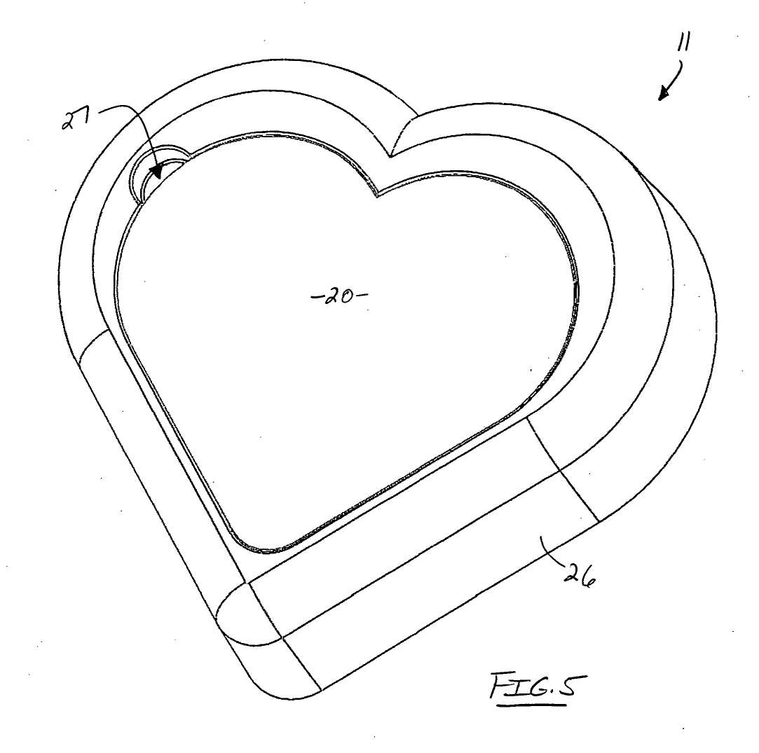

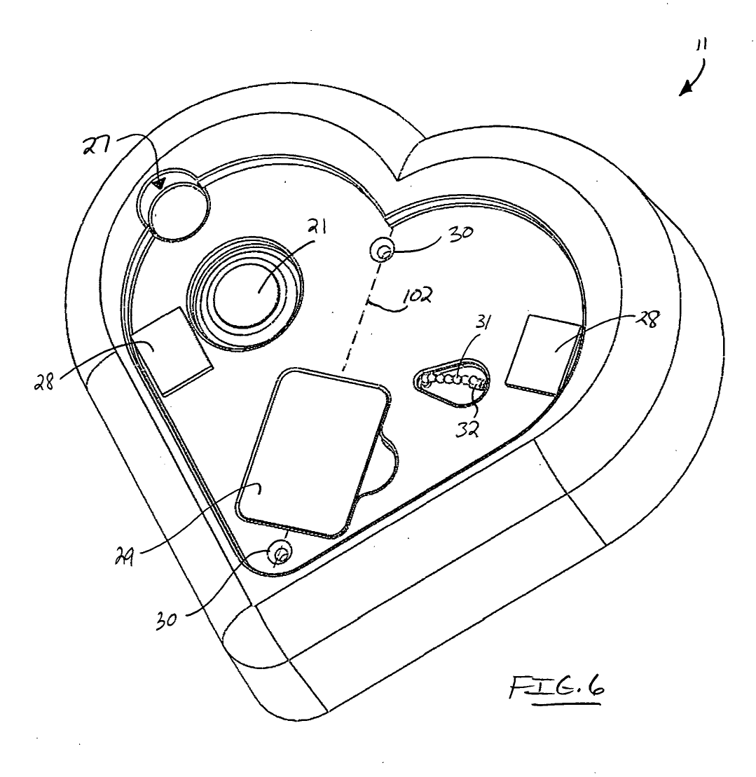

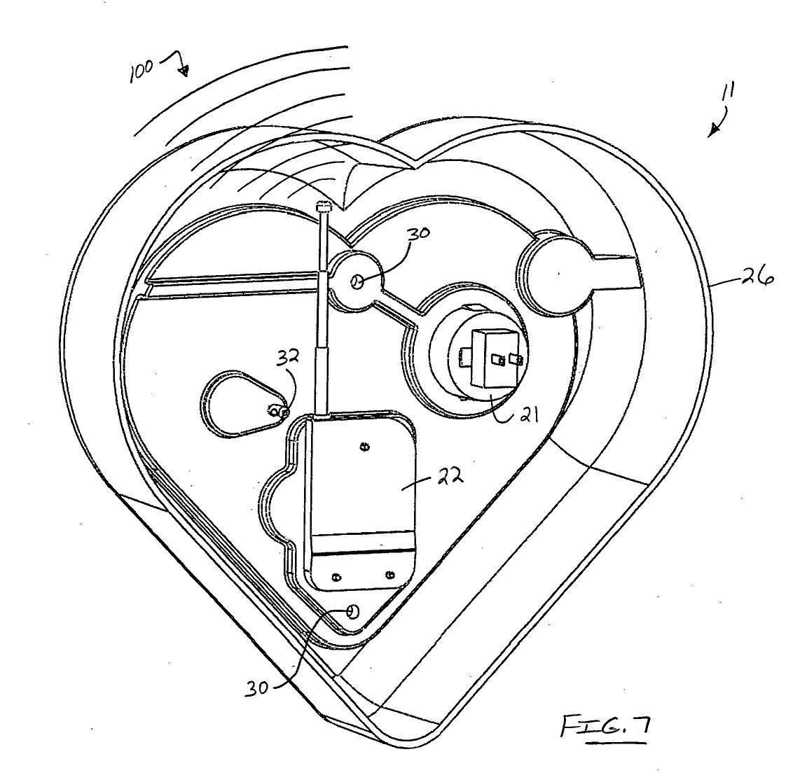

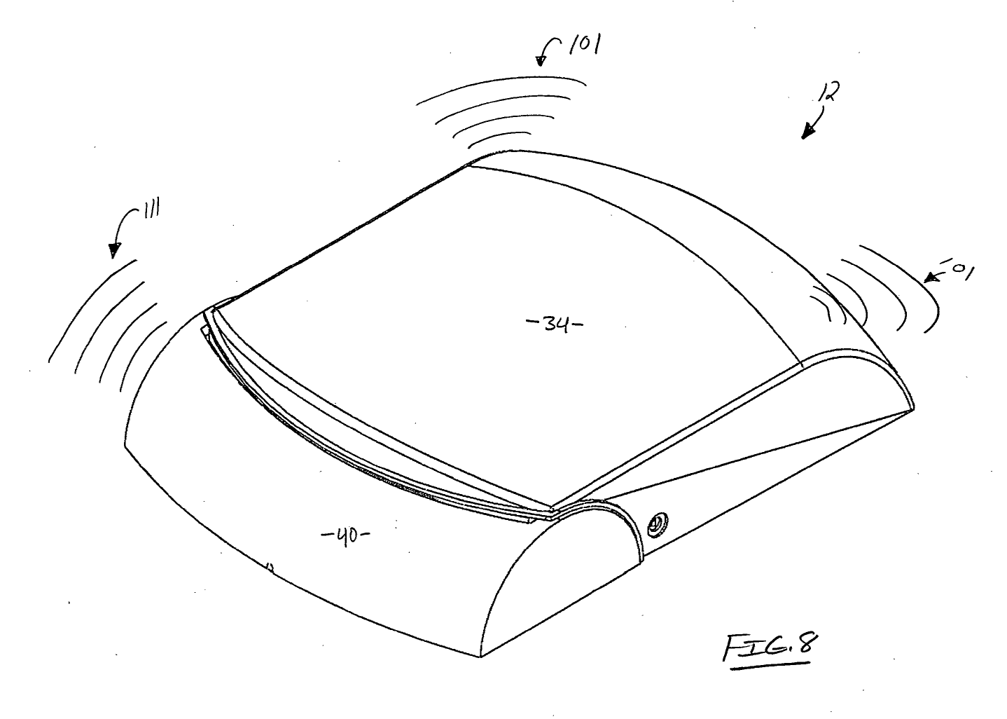

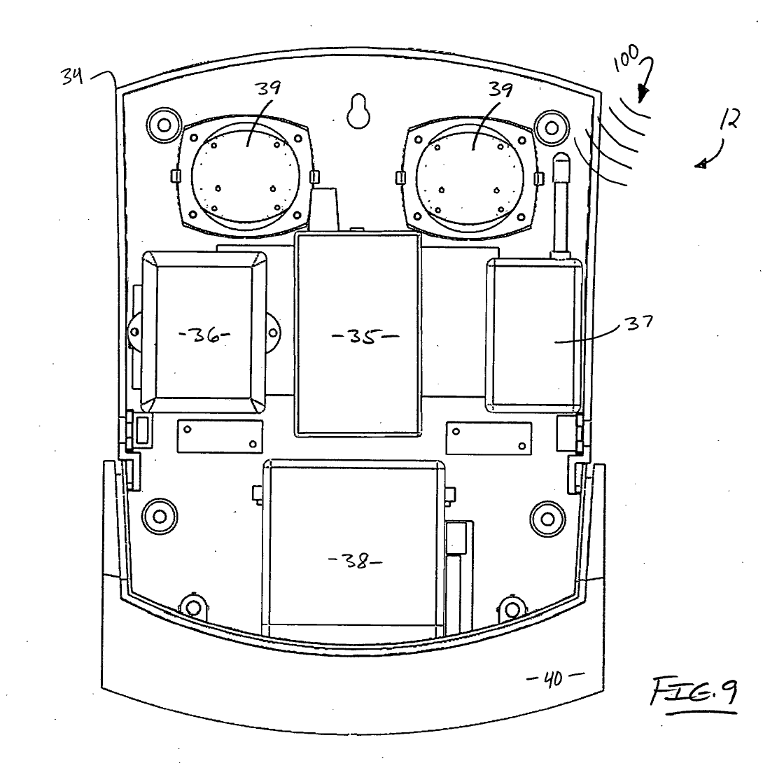

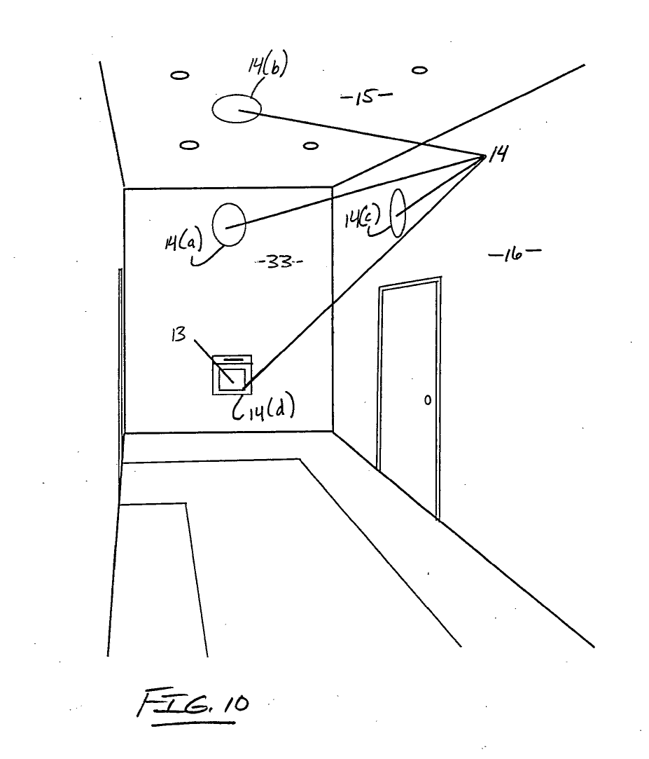

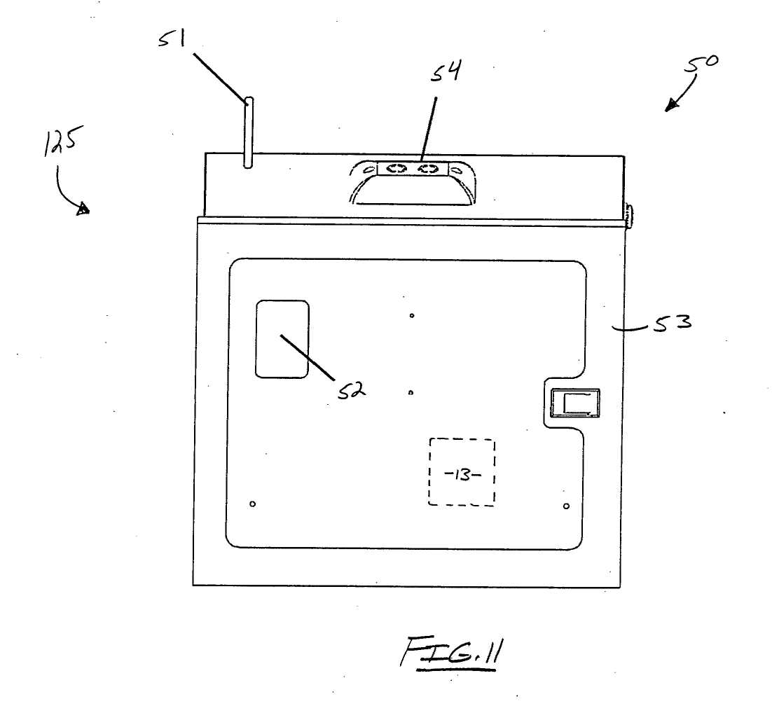

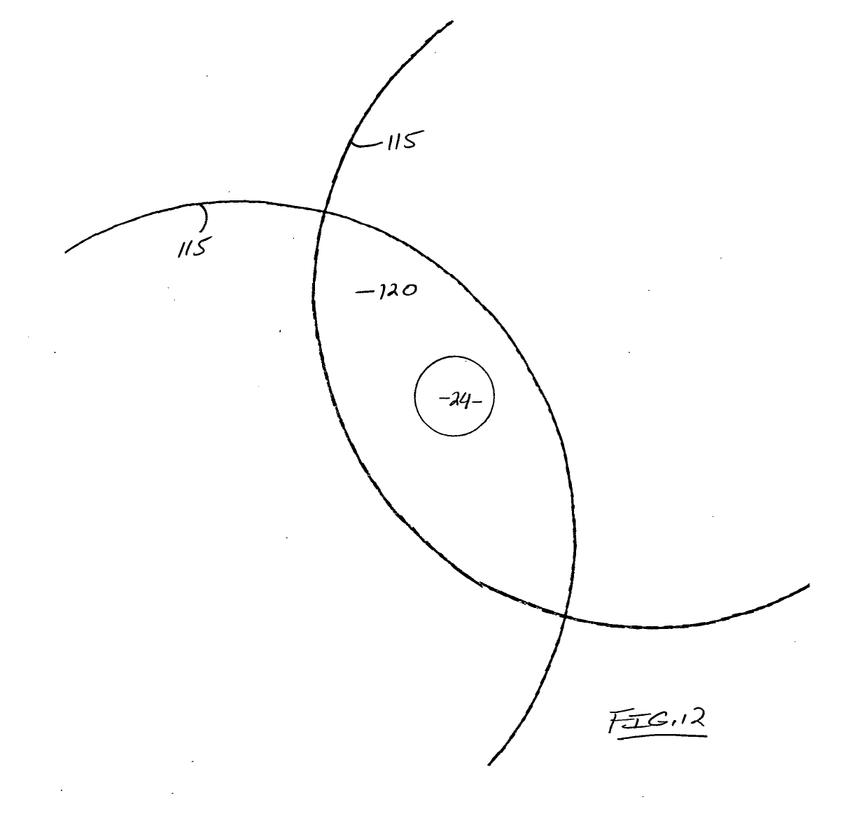

### BACKGROUND OF THE INVENTION

1. Field of Invention

The present invention generally relates to field of medical response systems, and more particularly to developments for rapidly identifying the location of Automatic External Defibrillators (AED's) for timely treatment during cardiac emergencies.

2. Description of the Prior Art

The prior art shows various article-locating systems incorporating the use of transmitters and receivers, the latter of which may be coupled or otherwise affiliated with various articles of manufacture. When lost or misplaced, the user may operate a transmitter for sending a signal to the receiver for triggering an alarm to locate the lost or misplaced article of manufacture. In this regard, it is noted that items such as luggage, vehicles, skis, animals, etc. can all be outfitted with a receiver and alarm for providing some perceptible stimulus for enabling the user to locate the host article.

It is further noted that fire protection systems, when outfitted upon a building structure may well comprise various means for activating a fire alarm, which when activated, trigger alarms spaced throughout the building at sites most conducive to alerting persons within (auditory) range of the alarm that a possible fire scenario is then present. It is noted that the means for activating fire alarms may be wireless and singularly trip multiple alarms throughout the facility. The key aspect of fire alarm systems, however, is there are typically a plurality of audible alarm devices spaced throughout a building that function to essentially warn persons to vacate the premises. The evacuation route is typically the nearest exit. The audible system, however, does not direct or guide persons to the nearest exits, but rather provides a general alarm or warning that persons should vacate the premises (via whatever route).

The most notable art for directing cardiac emergency responders to an AED necessarily differ from the foregoing types of art in several ways. Several of the more pertinent patent disclosures relating to this type of art are briefly described hereinafter. U.S. Pat. No. 6,292,687 ('687 Patent), which issued to Lowell et al., discloses a Medical Emergency Response and Locating System.

The '687 Patent describes an emergency response system for detecting, locating, and responding to a predetermined medical emergency, such as sudden cardiac arrest/sudden cardiac death, in a person being sensed for the predetermined emergency, and wherein the medical emergency can be treated with portable medical equipment, such as an AED machine for treating cardiac arrest/sudden cardiac death.

The system comprises a reader worn by the person being sensed for reading a dysfunction indicating the existence or imminent existence of the emergency condition and a sensor for determining when an emergency condition is read and producing an alarm signal. A processor activates a personal alarm at the location of the person suffering the emergency, now the victim, which indicates the emergency and the victim's location to those in the victim's immediate area.

The processor also transmits an alarm signal to an alarm indicator on the portable medical equipment to alert anyone in the immediate area of the equipment (i.e. an emergency response person) that a victim is in immediate need of such equipment. Preferably, the alarm signal includes location signals which indicate the location of the victim to direct the emergency response person with the equipment to the victim. The processor preferably also transmits or causes transmission of an alarm signal to a remote emergency response center which receives the alarm and dispatches an emergency response person or emergency response team to the victim.

Again, location information in or with the alarm signal directs the emergency response person to the location of the victim. The invention provides a closed loop system, i.e., victim emergency, transmission of alarm signal and location information to an emergency response person, and response by an emergency response person to the victim.

It will be noted from a consideration of the '687 Patent that although the system functions to detect, locate, and respond to a person in cardiac arrest, it requires certain means for sensing the cardiac arrest and a circuitry that responds to the sensor, hence activating an alarm on the AED. This approach does not make the locating mechanism available to a person that does not have sensing means located on oneself. It is intended for persons with known heart conditions and does not aid a rescuer in locating an AED for a victim without this sensing means.

U.S. Pat. No. 6,493,581 ('581 Patent), which issued to Russell, discloses a System and Method for Rapid Recruitment of Widely Distributed Easily Operated Automatic External Defibrillators. The '581 Patent describes a system for rapid recruitment of AED's and operators of the AED's comprising a plurality of AED's. A locator is operable to determine locations of the defibrillators, a victim, and a potential operator of the defibrillators. A communicator is operable to communicate the location of at least one of the defibrillators to the potential operator.

Notably, the system requires that the victim's location be entered into a system capable of evaluating stored data for locating trained operators and AED's. This system requires certain means for entering a victim's absolute position. This may be via a cell phone or device for entering an address. This system is complex and relies on a network to make operable.

U.S. Pat. No. 6,937,150 ('150 Patent), which issued to Medema, et al., discloses a Method and System for Locating a Portable Medical Device. The '150 Patent describes a wireless automatic location identification (ALI) capable system comprising a medical device having a wireless data communicator, a wireless communication network, and a remote locating service for remotely locating and monitoring one or more medical devices over the wireless communication network.

When the medical device is linked to the remote locating service over the communication network, the ALI-capable system identifies the location of the medical device and relays the location information to the remote locating service. The system permits reliable determination of the location of the medical device wherever the medical device is situated. The medical device may further be configured to transmit signals indicative of its status, condition, or self-test results, to the remote locating service. This feature allows the remote locating service to centrally monitor the status or condition of a plurality of medical devices.

The '150 Patent describes a system comprising a medical device with wireless communications, a wireless network, and a remote locating service. Notably, this system requires that a monitoring system be notified before the process of locating a local medical device can be completed. This system locates the medical device only after the device has been found and put to use by the rescuer. In other words, the locating device is to notify and aid rescuers in finding the victim, not to locate the actual medical device for implementation.

The foregoing systems have been unsuccessful due, in large measure to the fact that AED's are oftentimes not registered and the difficulty in obtaining funding from the monitoring services. Large scale participation is required before they will be successful. The prior art thus perceives a need for a system that is operational on a small scale, is local, and does not require mass participation.

The prior art further perceives a need for a system that simply provides the position and/or location of an AED relative to a rescuer and/or responder without requiring the sporadic availability of cell phones nor the necessity to enter an address. Further, the prior art perceives a need for an AED locating system that does not require the time and expense of tapping into a monitoring network.

### SUMMARY OF THE INVENTION

Thus, it is a primary object of the present invention to provide a local system that does not require the use of a monitoring network or database of stored devices. The system according to the present invention first increases awareness of on-site AED's by the presence of specifically colored/shaped transmitter locating stations. In this regard, the system preferably incorporates the use of red, heart-shaped transmitter locating stations. Thus, as potential responders enter a facility a red heart-shaped transmitter station provides an immediate visual indication that AED's are present within the entered facility.

These red heart-shaped transmitter stations are ideally positioned within the facility by way of codes established per the National Fire Protection Association (NFPA) 72 guidelines for placement of fire manual call stations. This means that the red, heart-shaped transmitter stations should be positioned within 5 feet of every exit and in multi-story buildings at elevators and stairways. This will allow anyone in the facility to quickly come across a red, heart-shaped transmitter station.

The red, heart-shaped transmitter stations, while serving to promote defibrillation awareness, may thus be quickly located and essentially function to provide a two-stage or three-stage call station. In two-stage versions, the responder must first flip open the heart insert and then press the button behind the heart-shaped cover insert in order to activate the transmitter.

In the three-stage versions, the responder must also break or snap a tamper-proof tie, which must first be broken before the responder may access the call button behind the heart-shaped insert as in the two-stage version. It is contemplated that the multiple stages function to minimize tampering and false triggers.

Upon pressing the signal-transmission button (located beneath or behind the heart shaped cover insert) a wireless signal is transmitted to a receiver station. It is contemplated that receiver stations may preferably be located within 1,000 feet line of site or less as dictated by the number of walls, corners, and/or objects situated intermediate the transmitter and receiver stations. Upon receiving the transmitted signal the receiver preferably activates an audible (and visual) alarm for guiding responder(s) toward the alarm/AED.

In this last regard, it should be noted that the receiver station and alarm unit are positioned near the AED site, but in a relatively higher elevation as compared to the AED for enhancing transmission of the audible alarm over the heads of people or over obstacles that may be present in the line of path between the locator transmitter station and the receiver/alarm station.

It is contemplated that the audible alarm will be of sufficient intensity (e.g. >120 decibels) to travel to the locator transmitter station/site such that the signal sender may be able to aurally perceive the audible alarm. The responders may thus quickly follow the guiding alarm to/toward the AED. It is a very natural ability for a listener to quickly make decisions on corridor junctions to determine the most direct path to a high intensity alarm. By following the audible alarm, the listener is quickly directed to the AED.

It is further contemplated multiple transmitters will increase the ability to quickly locate a specifically positioned AED by nearly the same factor of added sporadically positioned AED's, at a fraction of the expense to install numerous AED's. It is further contemplated that the transmitters will have sufficient signal range that will match the alarm audible transmission range such that the responder can clearly identify the alarm over local noise and rapidly follow the audible to/toward the AED.

The signal-sending button at the transmitter station may preferably be a momentary contact push button. The push button will activate a transmitter that provides an output signal that is unique to the device and easily recognized as received by the receiving unit. Multiple transmitter stations may be programmed to specific receiver unit(s) and thus transmitter units may be grouped with specific receiver units. This will allow multiple systems to operate within a close proximity and assure that the responder is directed to the most convenient AED.

The receiver unit may be positioned within the AED cabinet, as part of AED signage above the AED, or a stand alone separate device mounted near the AED. The receiver will be preferably battery-powered, however may be line wired. The receiver would preferably be capable of activating an alarm for a timed period and then shut off and be ready for the next trigger. The activation time must be sufficient for the time it takes to travel from the farthest transmitter to the receiver/AED. It is suggested that this may be less than 3 minutes.

It is further suggested that the receiver be capable of operating in a test mode such that during testing of the transmitters, only minimal aural disturbance is realized. This may be done through holding the transmitter button for an extended period where the receiver checks for the transmit signal after a period of time and if still set turns the alarm off. Another approach is to include a test switch on the receiver box or for a second (test) button at the transmitter.

### BRIEF DESCRIPTION OF THE DRAWINGS

Other features of our invention will become more evident from a consideration of the following brief description of patent drawings:

Figure No. 1 is a schematic top plan type depiction of a facility layout showing rooms, corridors, and doorways with periodically positioned heart-shaped transmitter devices according to the present invention and receiver/alarm device(s) pair-positioned with automatic external defibrillator device(s) according to the present invention.

Figure No. 2 is a schematic top plan type depiction of the facility layout otherwise depicted in Figure No. 1 showing auditory alarm ranges emanating from the positioned receiver/alarm device(s) pair-positioned within visual proximity to the automatic defibrillator device(s) according to the present invention.

Figure No. 3 is an enlarged fragmentary top plan type depiction of a facility layout showing a receiver/alarm device pair-positioned in superior adjacency to an automatic defibrillator device as attached to a wall adjacent a structural corner.

Figure No. 4 is a frontal perspective view of a exit doorway of a facility with a heart-shaped transmitter device according to the present invention shown positioned adjacent the exit doorway.

Figure No. 5 is a frontal perspective view of a heart-shaped transmitter device according to the present invention with removable cover in positioned placement to cover a signal activation button.

Figure No. 6 is a frontal perspective view of a heart-shaped transmitter device according to the present invention with removable cover removed to show otherwise hidden structure inclusive of a signal activation button.

Figure No. 7 is a rear perspective view of a heart-shaped transmitter device according to the present invention depicting a transmitter unit sending a wireless signal.

Figure No. 8 is a frontal perspective view of a receiver/alarm device according to the present invention depicting audio and visual signals emanating from the device.

Figure No. 9 is a rear plan view of the receiver/alarm device according to the present invention depicting a receiver unit receiving a wireless signal with various alarm-supporting components in communication with the receiver unit.

Figure No. 10 is a depiction of a responder's viewpoint looking down a corridor depicting alternative placement locations for receiver/alarm devices according to the present invention relative to an automatic external defibrillator location.

Figure No. 11 is a front plan view of a singular alternative device or unit according to the present invention which device functions to (1) store an automatic external defibrillator unit, (2) receive wireless signals, and (3) provide alarm signal for guiding would-be responders toward the device.

Figure No. 12 is a diagrammatic depiction of overlapping signals at a receiver/alarm site thereby depicting the ability of multiple transmitter devices according to the present invention to activate a single receiver/alarm site for guiding would-be responders towards the site.

### DESCRIPTION OF THE PREFERRED SYSTEM/METHODOLOGY

The placement of Automatic External Defibrillators (AED's) throughout schools, health clubs, businesses, airports, shopping malls etcetera has shown demonstrative effects on life savings by trained as well as lay persons. It perhaps goes without saying that early defibrillation is critical to the survival of cardiac arrest victims, and thus the rapid availability of these AED's is of utmost importance.

Financial constraints, however, often do not allow for high density placement of AED's. A lay rescuer unfamiliar with a facility may be standing within feet of an AED located around a corner and headed in a direction away from the closest AED when desirous of locating an AED. Labyrinth like hallways, corners, corridors, and doors may obstruct an AED from rapid locating. Due to the relatively high cost of AED's it is often not financially feasible to locate them as required for rapid identification and retrieval, therefore the prior art perceives a need for the provision of certain low cost means to aide in locating these devices.

The system(s) and method(s) according to the present invention thus support AED awareness and accessibility by boldly announcing the presence and whereabouts of AED's in a facility. In this regard, the reader is directed to the generic facility or building layout 10 as generally depicted in Figure Nos. 1 and 2. Referencing Figure No. 1, it will be seen that a number of heart shaped, transmitting devices 11 are mounted in numerous strategic locations such as entrances of public facilities, as per NFPA requirements/standards for fire alarm pull/activation devices.

Second, upon manual activation in a cardiac emergency these heart shaped transmitting devices 11 transmit a signal to a receiver device 12 that preferably activates an (audible) alarm near the AED as depicted and referenced at 13. The responder or rescuer is thereby enabled to rapidly follow the (audible) alarm to the location of the AED 13. The receiver device may be preferably located at any number of locations (as at 14) in the adjacent, visually perceptible vicinity of the AED 13 as generally depicted in Figure No. 10. Referencing Figure No. 10, it should be noted that one such location 14 may be within the AED 13 (typically stored or housed within a storage unit).

The need to shorten AED response time cannot be overstated, and thus the ability to quickly find an AED 13 within a building or facility 10 as generically depicted in Figure No. 1 can be daunting and untimely managed if sufficient guidance is not provided. Studies have shown that in a cardiac arrest scenario (in which oxygenated blood flow stops), brain cells begin to die after roughly 4-6 minutes. Following the initial 4-6 minute period, the brain is destroyed at a rate of about 20% per minute.

Accordingly, defibrillation via an AED 13 should be applied within 5 minutes for optimal results, it being understood that after about 10 minutes the chances of a successful revival are drastically reduced. Given that the average response time by emergency medical services in the United States is roughly 9 minutes, the need for means for quickly guiding a responder to an AED 13 is easily understood.

Tests have shown that a brisk walk rate is roughly 350 feet per minute, while a more typical walk rate in search for an AED 13 has been measured to be roughly 250 feet per minute. Further, in a typical school hallway a 120 decibel (dB) alarm can be readily heard by a responder (of average aural ability) at a distance of roughly 300 feet when sourced from within a labyrinth type, multiple corner-corridor facility 10; and roughly 600 feet when sourced in a straight-line path.

It is further noted that at hallway junctions, the human ear and brain readily distinguish the shortest path to an audible alarm by easily determining the direction of loudest sound or best preserved sound waves. The typical measured line of sight distance for identifying a well marked AED 13 is about 200 feet, and commonly available radio frequency signals can radiate beyond 300 feet through obstacles.

Given the criticality of time-based responses to cardiac emergencies, a few AED locator or guidance scenarios bear mention. For example, if an AED 13 were to be placed in a central location with no remote AED locator transmission device 11 more than 300 feet away from the AED 13.

The worst case scenario might be that a cardiac arrest victim drops directly around the corner from an AED 13 at which time the rescuer/responder does not see the AED 13 and heads off in a direction away from the AED 13. The responder in such a scenario would be required to travel 300 feet to the locator device (known to the responder) plus (+) 300 feet back to the AED 13.

The responder would then retrieve the AED 13 and return to the patient directly around the corner. The total distance traveled in this scenario would be roughly 600 feet and on a typical travel rate would take under 2.5 minutes. If the locator transmission devices 11 were not present, the rescuer/responder could easily make multiple wrong turns and try multiple corridors nearly consuming the entire 10 minutes. With locator transmission devices 11 positioned preferably no more than 300 feet from the AED 13, rescuers/responders should be able to initiate use of the AED 13 within about 2.5 minutes.

The time taken by a rescuer/responder to expose a patient's chest, shave if necessary, apply pads, and initiate defibrillation is estimated to take from 30 seconds to 2 minutes depending on the rescuer's/responder's familiarity with AED's. If the rescuer/responder were at the same location with the patient at the time of cardiac arrest, defibrillation could conceivably be initiated well within the critical 5 minutes.

According to the present system and methodology, the rescuer/responder follows the audible alarm toward the receiver or alarming device 12. The AED 13 must be readily visible as the responder nears the alarming device 12, as exemplified by placing the AED 13 directly underneath an alarming device such that a responder standing directly below the alarming device 12 may come face to face with the AED 13 as generally depicted in Figure No. 3, and referencing the receiver location 14(a) relative to the AED 13 in Figure No. 10.

It is recommended that the receiver/alarm device 12 be mounted a maximum distance of 15 feet horizontally from the AED 13, however should be mounted as close to the ceiling as possible, and must be in line of sight. Referencing Figure No. 3, it will be seen that the area bound at 110 depicts a maximum 15 feet and line of sight placement for the locator receiver device 12 as compared to AED location. Sound waves, it is noted, will consistently travel better near the ceiling due to the fact that people and/or other obstructions in the hallways tend to attenuate the sound waves.

The preferred location for mounting the receiver/alarm device 12 is directly above the AED 13, near the ceiling 15 as at location 14(a), but alternative locations: 14(b) (on the ceiling 15), 14(c) (on an adjacent wall 16), or 14(d) (within the AED (storage cabinet)) are also contemplated so long as the responder may visually perceive the AED 13 when approaching the receiver/alarm device 12.

It is further recommended (as per National Fire Protection Association (NFPA) 72 standards) that the locator transmitters or transmission devices 11 be installed within 5 feet of entrances/exits 17 as generally depicted in Figure No. 4; near elevators; and stairways. In addition, adjoining corridors or common areas where decisions must be made as to a direction to travel to locate and AED 13 are good spots to install the heart-shaped transmitters 11. These transmitter devices 11 are preferably installed with the top of the heart at 48 inches above the floor. This is a maximum height per the standards set forth in the Americans with Disabilities Act of 1990 (ADA), although higher positions are typically better for visibility and convenience of the non-disabled.

In the event of a cardiac emergency, the order of required actions differs depending on whether a lay or trained rescuer responds, as well as the physical conditions of the victim. To simplify the scenarios, it may be assumed that a lay rescuer experiences the unfortunate and sudden collapse of a victim. Firstly, the rescuer/responder may dial 9-1-1. Secondly, if the rescuer/responder have good reason to believe an AED 13 is on sight (e.g. they saw a red, heart-shaped transmitter device 11 when entering the facility 10, the rescuer/responder may retrieve the AED 13 via the transmitter device 11.

The rescuer/responder will likely encounter a red, heart-shaped locator transmitter device 11 due to the relatively high density of devices 11 positioned within the facility 10 relative to the AED's 13 as generally depicted in Figure No. 1. Upon locating a transmitter device 11, the rescuer/responder may pull open the cover 20 and press the transmitter activation button 21, which button 21 is in electrical communication with a transmitter 22.

The transmitter activation button 21 activates the transmitter 22, which then sends a radiating (radio frequency) signal 100 toward the receiver/alarm device 12 tuned for receiving the signal 100. Upon receiving the activating signal 100, the receiver/alarm device 12 activates an audible alarm signal as at 101. The rescuer/responder may then follow the audible alarm to the AED 13. Upon approaching the alarm device 12, the rescuer/responder visually perceives the AED 13 adjacent the device 12 and retrieves the AED 13. Finally, the rescuer/responder may return to the victim with the AED 13 in hand, and may administer defibrillation and possibly other necessary treatments (e.g. cardio pulmonary resuscitation (CPR)).

Referring now to the drawings with more specificity, the preferred system and/or methodology according to the present invention generally concerns a defibrillator locating system/method for enabling would-be defibrillator users to quickly identify defibrillator location and carry a located defibrillator or AED 13 to a cardiac emergency site. The system may be said to comprise, in combination: at least one (signal) transmitting device as at 11, a (signal) receiving device as at 12, and a defibrillator or AED as at 13, all as outfitted within a facility as at 10 in Figure No. 1.

It will be noted from an inspection of Figure No. 1 that a plurality of transmitter devices 11 are positioned at key locations within the facility 10. It will be recalled that the locations for the transmitter devices 11 may be dictated or suggested by the NFPA 72standards for fire alarm activation devices. Figure No. 1 depicts ten transmitter device site locations, some of which are in closer proximity to a first receiver-alarm device/AED site as at 24, and some of which are closer to a second receiver alarm device/AED site as at 25. It is contemplated that upon activation, the activated transmitter device 11 will activate the closest receiver/alarm device 12 for directing the responder to the closest AED 13.

Each signal-transmitting device 11 is preferably attached to a vertical transmitter support structure such as a wall 23 at a transmitter site. Each signal-transmitting device 11 preferably comprises a red, heart-shaped housing 26 for quickly drawing a responder's attention toward the device 11. In this regard, it is contemplated that a red, heart-shaped transmitter device 11 is themed for cardiac emergencies and thus is more apt to quickly draw one's attention toward the device 11 for its primary purpose. Devices taking on other shapes, it is believed may well delay recognition time.

In this last regard, the reader is directed to U.S. patent application Ser. No. 12/459,960, filed in the United States Patent and Trademark Office on 09 Jul. 2009, which application discusses in greater depth the benefits of heart-shaped storage units themed for cardiac emergencies. The specifications of U.S. patent application Ser. No. 12/459,960 are hereby incorporated by reference thereto insofar as those specifications support the present system and method(s).

In addition to the housing 26, each transmitter device 11 further preferably comprises a removable cover as at 20; a finger-receiving slot 27 for enabling the responder to quickly manually insert one's fingertip underneath the cover 20 for its removal; low profile dual fastening structures 28 positioned laterally relative to the medial plane 102 of the housing 26 for removably retaining the cover 20 in a button-covering position; a battery (or power source/control panel) access panel as at 29; and mounting apertures 30 centered on the medial plane (as at 102) for mounting the device to a support structure such as a wall 23.

It is contemplated that the dual fastening structures 28 can be of a hook or loop type fastening structure with matable structure attached to the cover 20 for removably hook-fastening the cover 20 to the structures 28. Alternatively, the structures 28 can comprise female or male snap like structures with matable structure attached to the cover 20 for removably snap-fastening the cover 20 to the structures 28.

The heart-shaped cover 21 essentially functions to conceal the button 21 and thus provides two-stage activated for security, firstly removal of the cover 20, and secondly activation of the button 21. In other words, the button 21 cannot be activated unless the cover 20 is first removed. The finger slot 27 may further be outfitted with a tamper-resistant tie (not specifically illustrated) for tamper control.

Each transmitter device 11 further preferably comprises certain transmitter means and certain cover-retention means. The transmitter means essentially function to transmit a wireless signal as at 100 having a certain transmission signal range, and may be exemplified by a radio frequency signal transmitter unit 22. The certain cover-retention means essentially function to retaining the cover 21 adjacent the housing 26 after it has been removed so as to expose the button 21 (in electrical communication with the transmitter means (circuitry not specifically shown). It is contemplated that the cover-retention means may be exemplified by a chain 31 or similar other tether device the ends 32 of which are attached to the cover 21 and the housing 26.

The signal-receiving device(s) 12 are preferably attached to a receiver support structure such as a wall as at 33 (or adjacent wall 16, or ceiling 15). Each signal-receiving device 12 preferably comprises a receiver housing as at 34; a power supply 35 (preferably for converting 110-130 volt alternating current to 12 volt direct current), control relays 36, and circuitry (not specifically shown) for electrically communicating the components as primarily housed within a receiver control panel 38.

Each signal-receiving device 12 further preferably comprises certain signal-receiving means and certain auditory alarm means in electrical communication with the signal-receiving means. The signal-receiving means may be exemplified by a radio frequency signal receiver unit as at 37. The receiver support structure (e.g. wall 33) is preferably located within the transmitter signal range for enabling the receiver device 12 to receive a transmitted wireless signal 100 from the activated transmitting device 11 as generally depicted in Figure No. 9.

The auditory alarm means may be preferably exemplified by dual audible alarms (rated for at least 120 decibels) as at 39, and essentially function to provide an auditory signals 101 to emanate from an alarm site as at 24 or 25 as generally depicted in Figure No. 2. The area bound by signal 101 emanating from site 24 is contemplated to cover 300 feet obstructed, 600 feet line of sight, and the area bound by signal 101 emanating from site 25 is contemplated to cover 300 feet obstructed, 600 feet line of sight. The auditory signal(s) 101 preferably have a sufficient decibel level whereby a signal-sending user at the transmitter site may be able to aurally perceive the auditory signal 101.

Each receiver/alarm device 12 may be further outfitted with certain visual alarm means such as a strobe light and red lens 40 for flashing light as at 111 from the receiver/alarm device 12. It is contemplated that a strobe light may be cooperably associated with the control panel 38 such that light 111 may transmit through the red lens 40 for visually guiding responders toward the receiver and/or AED site. Thus, it is contemplated that each receiver device 12 may preferably comprise certain visual alarm means in communication with the signal-receiving means for providing a visual signal at the alarm site, which visual signal may well function to enhance the responder's ability to visually locate the defibrillator site via the alarm site.

The defibrillator or AED 13 is removably attached to a defibrillator support structure such as a wall 33 at a defibrillator site. The defibrillator site is visually perceptible in proximity to the alarm site such that when the auditory alarm means draw or guide a responder toward the alarm site, the responder may visually perceive the defibrillator site. The removably attached defibrillator 13 enables the responder to quickly carry said defibrillator to a cardiac emergency site for further use.

The transmitter site(s) and the receiver site are each preferably located within the facility 10 such that the signal-sending user/responder may be able to aurally perceive the audible signal 101 and thereby be audibly drawn or guided to the alarm site (e.g. site 24 or 25 as the case may be) from the selected transmitter site along a clear path via the aurally perceived auditory alarm. In other words, it is contemplated that the pathways leading from the transmitter site to the alarm site are clear pathways as generally depicted by broken lines 112 in Figure No. 1.

It is further contemplated that the defibrillator sites be positioned so that a single defibrillator site is visually perceptible from the alarm site. In this regard, it is contemplated that the receiver/alarm unit 12 may be cooperably included with the AED unit 13 as generally depicted in Figure No. 11. In this regard, a combination AED-receiver/alarm device 50 for providing an alarmed defibrillator site 125 is generally depicted in Figure No. 11. A receiver antenna 51, a receiver module 52, and an audible alarm means or structure 54 mounted to the AED storage cabinet 53.

The system according to the present invention preferably comprises a plurality of transmitting devices each of which are preferably attached to a vertical transmitter support structure at a respective transmitter site, which sites are spaced such that the respective wireless signals 115 each have a respective signal range. The respective signal ranges of the signals overlap as at 120 at the alarm site (e.g. sites 24 or 25) for enabling alarm activation via multiple transmitter site locations.

While the above description contains much specificity, this specificity should not be construed as limitations on the scope of the invention, but rather as an exemplification of the invention. For example, it is contemplated that the present invention essentially provides a defibrillator locating system for enabling would-be defibrillator users/responders to quickly identify a defibrillator storage location. The system may be said to comprise at least one transmitting device 11 and a receiving device as at 12.

Each transmitting device 11 is preferably attached to a vertical transmitter support structure at a transmitter site, and comprises means for transmitting a wireless signal having a signal range. The receiving device is preferably attached to a receiver support structure, and comprises certain signal-receiving means and certain auditory alarm means in communication with the signal-receiving means. The receiver support structure is located within the signal range for enabling the receiver device to receive a transmitted wireless signal from the transmitting device.

The auditory alarm means essentially function to provide an auditory signal 101 at an alarm site such as locations 24 or 25, which auditory signal 101 preferable has a sufficient intensity (i.e. decibel) level whereby a signal-sending user at the transmitter site may be able to aurally perceive the auditory signal 101. The auditory alarm means thus provide the essential and critical function of drawing and/or guiding a responder toward the alarm site such that the responder may visually perceive a defibrillator storage site at as generally depicted at 13.

When viewed from a methodological perspective, the present invention may be said to set forth certain methodology for locating a defibrillator, which method may be said to comprising the positioned placement of a defibrillator at a defibrillator storage site; at least one alarm at at least one alarm site; a signal receiver in communication with each alarm at a receiver site; and at least one signal transmitter at at least one transmitter site.

Each alarm site is visually perceptible from the defibrillator storage site, and each transmitter site has a certain transmitter-to-receiver site distance or range. A signal may then be activated at a select transmitter site, which signal has a signal range greater than a respective transmitter-to-receiver site distance. The signal is then received at the receiver site via the receiver, and an alarm stimulus is activated via the communicating receiver and alarm. The alarm stimulus may then be perceived by a responder thereby drawing and/or guiding the responder's attention toward the alarm stimulus for enabling the responder to visually perceive the defibrillator storage site from a respective alarm site.

At least two signal transmitters may be positioned at respective transmitter sites wherein each transmitter site has a transmitter-to-receiver site distance. The respective transmitter sites are spaced from one another such that respective signals each have a respective signal range that may overlap at each alarm site for enabling alarm activation via multiple transmitter site locations.

Accordingly, although the invention has been described by reference to certain preferred and alternative constructions, as well as certain methodology, it is not intended that the novel disclosures herein presented be limited thereby, but that modifications thereof are intended to be included as falling within the broad scope and spirit of the foregoing disclosure, the following claims and the appended drawings.

We claim:

1. A defibrillator locating system for enabling would-be defibrillator users to quickly identify defibrillator location and carry a located defibrillator to a cardiac emergency site, said system comprising, in combination:
at least one transmitting device, each transmitting device being attached to a transmitter support structure at a transmitter site, the transmitting device having means for transmitting a wireless signal, the wireless signal having a signal range;
a receiving device, the receiving device being attached to a receiver support structure, the receiving device comprising signal-receiving means and auditory alarm means in communication with the signal-receiving means, the receiver support structure being located within the signal range for enabling the receiver device to receive a transmitted wireless signal from the transmitting device, the auditory alarm means for providing an auditory signal at an alarm site, the auditory signal having a sufficient decibel level whereby a signal-sending user at the transmitter site may be able to aurally perceive the auditory signal; and
a defibrillator, the defibrillator being removably attached to a defibrillator support structure at a defibrillator site, the defibrillator site being visually perceptible in proximity to the alarm site, the auditory alarm means for drawing a responder toward the alarm site such that the responder may visually perceive the defibrillator site, the removably attached defibrillator for enabling the responder to quickly carry said defibrillator to a cardiac emergency site.

2. The system of claim 1 wherein the transmitter site and the receiver site are each located such that the signal-sending user may be audibly guided to the alarm site from the transmitter site along a clear path via the aurally perceived auditory signal.

3. The system of claim 1 wherein said defibrillator site is the only defibrillator site visually perceptible from the alarm site.

4. The system of claim 1 wherein the receiver device comprises visual alarm means in communication with the signal-receiving means, the visual alarm means for providing a visual signal at the alarm site, the visual signal for enhancing the responder's ability to visually locate the defibrillator site via the alarm site.

5. The system of claim 1 wherein the alarm site is positioned above the defibrillator site for enhancing the responder's ability to visually locate the defibrillator site via the alarm site.

6. The system of claim 5 wherein the alarm site is positioned vertically above the defibrillator site for enhancing the responder's ability to visually locate the defibrillator site via the alarm site.

7. The system of claim 1 comprising at least two transmitting devices, each transmitting device being attached to a transmitter support structure at a respective transmitter site, the respective transmitter sites being spaced such that the respective wireless signals each have a respective signal range, the respective signal ranges overlapping at the alarm site for enabling alarm activation via multiple transmitter site locations.

8. A defibrillator locating system for enabling would-be defibrillator users to quickly identify a defibrillator storage location, said system comprising:
at least one transmitting device, each transmitting device being attached to a transmitter support structure at a transmitter site, the transmitting device having means for transmitting a wireless signal, the wireless signal having a signal range; and
a receiving device, the receiving device being attached to a receiver support structure, the receiving device comprising signal-receiving means and auditory alarm means in communication with the signal-receiving means, the receiver support structure being located within the signal range for enabling the receiver device to receive a transmitted wireless signal from the transmitting device, the auditory alarm means for providing an auditory signal at an alarm site, the auditory signal having a sufficient decibel level whereby a signal-sending user at the transmitter site may be able to aurally perceive the auditory signal, the auditory alarm means thus for guiding a responder toward the alarm site such that the responder may visually perceive a defibrillator storage site.

9. The system of claim 8 wherein the transmitter site and the alarm site are located relative to one another such that the signal-sending user may be audibly guided to the alarm site from the transmitter site along a clear path via the aurally perceived auditory alarm.

10. The system of claim 8 wherein the defibrillator storage site is the only defibrillator storage site visually perceptible from the alarm site.

11. The system of claim 8 wherein the receiver device comprises visual alarm means in communication with the signal-receiving means, the visual alarm means for providing a visual signal at the alarm site, the visual signal for enhancing the responder's ability to visually locate the defibrillator storage site via the alarm site.

12. The system of claim 8 wherein the alarm site is positioned above the defibrillator storage site for enhancing the responder's ability to visually locate the defibrillator storage site via the alarm site.

13. The system of claim 8 comprising at least two transmitting devices, each transmitting device being attached to a transmitter support structure at a respective transmitter site, the respective transmitter sites being spaced such that the respective wireless signals each have a respective signal range, the respective signal ranges overlapping at the alarm site for enabling alarm activation via multiple transmitter site locations.

14. A method for locating a defibrillator, the method comprising the steps of:
positioning an alarm at an alarm site, the alarm site being visually perceptible from a defibrillator storage site;
positioning a signal receiver in communication with the alarm at a receiver site;
positioning a signal transmitter at a transmitter site;
activating a signal at the transmitter site;
receiving the signal at the receiver site via the signal receiver;
activating an alarm signal via the communicating receiver and alarm; and
perceiving the alarm signal by a responder thereby drawing the responder's attention toward the alarm signal for enabling the responder to visually perceive and thus locate the defibrillator storage site from the alarm site.

15. The method of claim 14 comprising the step of positioning at least two signal transmitters at respective transmitter sites, the respective transmitter sites being spaced from one another such that respective signals each have a respective signal range, the respective signal ranges overlapping at the alarm site for enabling alarm activation via multiple transmitter site locations.

16. The method of claim 14 wherein the alarm site and the receiver site are within an alarm-receiver site.

17. The method of claim 16 wherein the defibrillator storage site and alarm-receiver site are within an alarmed defibrillator site.

18. The method of claim 14 wherein the alarm signal comprises an aurally perceptible signal.

19. The method of claim 18 wherein the alarm signal comprises a visually perceptible signal.

20. The method of claim 14 comprising the step of positioning a defibrillator at a defibrillator storage site before positioning the alarm at the alarm site.

21. The method of claim 14 wherein the steps of positioning: (1) the alarm at the alarm site, and (2) the signal receiver at the receiver site are performed simultaneously.
